(19) United States
(12) Patent Application Publication
(10) Pub. No.: US 2025/0302377 A1
(43) Pub. Date: Oct. 2, 2025

(54) Biomedical Parameters Monitoring System for the Diagnosis of Sleep Disorders

(71) Applicant: OXAMA MEDICAL CORP, Dover, DE (US)

(72) Inventors: Vincenzo PALUMBO, Milano (IT); Marco MORELLI, Bareggio (Ml) (IT); Claudio MATTAVELLI, Usmate Velate (MB) (IT); Massimo MOI, Vimercate (MB) (IT); Stefano VERGA, Como (IT); Davide FERRARIO, Marnate (VA) (IT); Costantino SOTTANA, Vicenza (IT)

(21) Appl. No.: 18/863,118

(22) PCT Filed: May 6, 2022
(86) PCT No.: PCT/IB2022/054221

§ 371 Date: Nov. 5, 2024

(51) Int. Cl. A61B 5/00; A61B 5/0205; A61B 5/024; A61B 5/08; A61B 5/083; A61B 5/11; A61B 5/113; A61B 5/1455; A61B 5/256; A61B 5/369; A61B 5/398

(52) U.S. Cl. CPC A61B 5/4806; A61B 5/0006; A61B 5/0205; A61B 5/1114; A61B 5/1135; A61B 5/14552; A61B 5/256; A61B 5/369; A61B 5/398; A61B 5/6803; A61B 5/02438; A61B 5/0816; A61B 5/0836

(57) ABSTRACT

A biomedical parameters monitoring system for the diagnosis of sleep disorders provides a plurality of sensors reading a series of biometric parameters, one or more external devices collecting the data read by the sensors for making the diagnosis, and a facial support on which at least one sensor is mounted for reading the electroencephalographic signal and the electrooculography signal and a sensor for reading the electromyographic signal; the facial support has a central part, suitable for being located at the mouth and the nose, on which a sensor is mounted to read the respiration rhythm and the emitted carbon dioxide signal; each of the sensors is provided with a radio transceiver to transmit the reading data to said one or more external devices. The result is a monitoring system without wiring and efficient from a diagnostic point of view.

### BACKGROUND OF THE INVENTION

The subject of the present invention is a system for monitoring a series of biomedical parameters useful for studying and identifying sleep disorders.

### PRIOR ART

Sleep disorders affect many people, compromising, in some cases, the quality of their life and altering the normal physiological activities of their body. Lack of sleep can cause chronic fatigue, decreased attention and concentration and irritability. Furthermore, prolonged insomnia can have harmful effects on the health.

Sleep disorders primarily affect the ability to fall asleep and stay asleep. Poor sleep quality and quantity, therefore, inevitably compromise the quality of life and can lead to important health problems.

The most important sleep disorders are obstructive sleep apnea, pathological snoring, insomnia, daytime hypersomnia, narcolepsy, nocturnal epilepsy, and parasomnia.

To diagnose sleep disorders, an examination called polysomnography is carried out, which consists in simultaneously recording a plurality of physiological parameters during the night, such as brain activity, eye movements, muscle tone, oro-nasal flow, thoraco-abdominal movements and oxygen saturation.

Brain activity is recorded through the electroencephalogram (EEG) using external electrodes, and the information coming from the EEG is mainly used to distinguish the different sleep stages. The EEG recording also provides useful information on the integrity and development of the nervous system. To make EEG recordings repeatable and comparable in the same patient and in different patients, the electrodes are positioned according to an international standard.

Eye movements in the various sleep stages are recorded by means of the electrooculogram (EOG).

Muscle tone is recorded through the electromyography (EMG). Although the EMG during sleep can be recorded by any group of skeletal muscles, it is now consolidated practice to use the submental muscles (mylohyoid muscle) to assess muscle tone. The EMG, in addition to being useful for studying the various sleep stages, provides important information for the evaluation of stress responses and with regard to movements.

The airflow to the nose and mouth is commonly recorded by means of a thermocouple or a thermistor placed directly near each nostril and the mouth, or by means of a nasal cannula connected to a thermocouple placed inside a control unit positioned on the chest.

The movements of the chest and the abdomen can be recorded by impedance or inductance plethysmography, pneumatic transducers, strain gauges, intercostal EMG.

The oxygen saturation (SPO2) is recorded by means of a pulse oximeter on a finger of the hand; this method represents the standard for continuous non-invasive evaluation of arterial oxygen saturation and of heart rate and rhythm.

The polysomnography is carried out using a special equipment just called polysomnograph, commonly consisting of several sensors and electrodes connected via numerous cables to one or more control units for processing and recording the related signals. In particular, at least three electrodes and respective cables are required for the EEG signal, two other electrodes and respective cables for the EOG signal, and two other electrodes and related cables for the EMG signal. In addition, a cable is provided for the oximetry signal, a cable for the thermocouple (if placed near each nostril and the mouth), and two cables for the movements of the chest and the abdomen. The disclosed polysomnograph is therefore provided with at least eleven cables which refer to a control unit which can be positioned on the patient's body or in some cases to a control unit near the bed.

It is clear that the polysomnograph necessarily requires the presence of medical or nursing staff for the correct positioning of the sensors and cables, but, above all, that the complex cable system in fact limits or prevents normal movements during sleep, disturbing the same and the neutrality of the sleep test. Further, the same test is frequently affected by errors due to the displacement of the sensors and of the electrodes owing to the traction of the cables. Therefore, there is a risk of failing in the realistic simulation of the patient's sleep and in the correctness of the test, and this is undoubtedly a big limit.

A further limit of current polysomnographs is the absence of non-invasive measuring of concentration of carbon dioxide (CO2) exhaled, both in numerical form (capnometry) and through the graphic expression of its trend over time (capnography). The assessment of the CO2 partial pressure concentration provides important information on ventilation (elimination of CO2 from the pulmonary system), perfusion (transport of the CO2 through the vascular system) and metabolism (production of CO2 by cell metabolism).

### OBJECT OF THE INVENTION

The object of the present invention is to propose a system for monitoring biomedical parameters for the diagnosis of sleep disorders that can overcome the limitations of prior art polysomnographs seen above.

### BRIEF DESCRIPTION OF THE INVENTION

This object is achieved by a biomedical parameters monitoring system for the diagnosis of sleep disorders according to the first claim.

### BRIEF DESCRIPTION OF THE DRAWINGS

To better understand the invention, a description of a non-limiting exemplary embodiment thereof is given below, with the aid of the attached drawings in which:

FIG. 1 shows a known polysomnographic system applied to a patient;

FIG. 2 shows a further known polysomnographic system applied to a patient;

FIG. 3 shows a biomedical parameters monitoring system for the diagnosis of sleep disorders according to the present invention, applied to a patient;

FIGS. 4,5 show, in a frontal and side view respectively, a component with sensors of the monitoring system of FIG. 3, applied to the patient's face;

FIG. 6 shows, in an enlarged front view, the component of FIGS. 4,5;

FIG. 7 shows a series of exploded sectional views, according to different section planes, of the component with sensors of FIG. 6;

FIGS. 8-13 show the sensors of the component with sensors of FIG. 6, each sensor being represented by means of a bottom view, a section view and a block circuit diagram.

### DETAILED DESCRIPTION OF THE INVENTION

In the known polysomnographic system of FIG. 1, for the diagnosis of sleep disorders a series of sensors and electrodes is used, connected by cables to different control units for signal processing.

During the sleep test, generally carried out in hospitals, a patient 1 is lying on a bed 2. Electrodes 3 are positioned on the head of the patient 1 for reading the electroencephalographic signal (EEG) and the electrooculography signal (EOG). The aforesaid electrodes are connected by cables 5 to a control unit 6 for signal processing. Furthermore, electrodes 4 are placed on the patient's chin to collect the electromyography signal (EMG), also connected by other cables 5 to the control unit 6.

The nasal respiratory flow is collected by means of a nasal cannula 7 and is conveyed by a flexible tube 8 to a thermocouple 9, which supplies information exclusively on the temperature of the inhaled or exhaled air and is connected to a control unit 13. The same control unit 13 also receives signals coming from a thoracic expansion sensor 10, from an abdominal expansion sensor 11, and from a pulse oximetry sensor 12. The connections to the control unit 13 are made by means of cables 14.

The control units 6 and 13 are in turn connected by means of cables 15 to a central processing and recording unit 16.

The known polysomnographic system of FIG. 2 is instead only partially wired and is generally used not only in hospitals but also in the patient's home. The partially wired system of FIG. 2 is equivalent to the fully wired system of FIG. 1 as regards the sensors and electrodes and the related wiring to the control units 6 and 13, with the difference that the latter are not physically connected to the central unit 16, but communicate with it by means of radio signals, or internally record the collected data, which are subsequently extracted by specialized medical personnel.

As previously explained, as both the systems disclosed in FIGS. 1,2 are provided with a complex wiring which in fact limits or prevents normal movements during sleep, perturbing the same and thus invalidating the neutrality of the sleep test, therefore they can be not very objective and are frequently affected by errors due to the displacement of the sensors owing to the traction of the cables.

With reference to the biomedical parameters monitoring system for the diagnosis of sleep disorders according to the invention of FIG. 3, also in this case the patient 1 lies on the bed 2, for example in a hospital or in outpatient clinics or also at the patient's home, both during single diagnostic events and during prolonged monitoring periods. Multiple sensors are positioned on the patient's body for reading the various biomedical parameters, for example, in a non-exhaustive manner, a sensor 20 for reading the EEG signal and the EOG signal, a sensor 21 for reading the EMG signal, a sensor 22 for reading the oximetry signal and for reading movement, a sensor 23 for reading respiratory rhythm and the signal of the carbon dioxide (CO2) emitted, a sensor 24 for reading respiratory sound and for reproducing sound or voice messages, a sensor 25 for measuring thoracic expansion, a sensor 26 for measuring abdominal expansion, and sensors 27,28 for measuring limb movement. Each of the aforesaid sensors is independent from the others, and can communicate by radio signal with a central system, such as for example a smartphone 30, or a modem 31, or a personal computer 32, etc.

The monitoring system of FIG. 3 provides a facial support 40, suitable for being positioned and fixed on the patient's face, on which the sensors 20-24 are mounted together with the electrodes for reading the EEG, EOG, EMG signals.

The facial support 40 is shown in detail in FIGS. 4-7 and substantially has a mask configuration. The facial support 40 can be made of soft elastic material, for example of silicone rubber or of any elastic polymer, or other, and can be of a low thickness, so as to be comfortable for the patient and adhere thanks to its elasticity to the face of the patient.

In the facial support 40, pockets are formed inside which the sensors 20-24 are inserted.

The sensor 20 for reading the EEG/EOG signal is inserted into a pocket 41, and the sensor 21 for reading the EMG signal is inserted into a pocket 48. The facial support 40 also incorporates electrodes 42,43,44 for measuring the EEG signal, electrodes 45,46,47 for measuring the EOG signal, and electrodes 49,50 for measuring the EMG signal.

The sensor 22 for reading the oximetry signal is inserted into a pocket 51, the sensor 24 for reading respiratory sound and for reproducing sound or voice messages is inserted into a pocket 52, and the sensor 23 for reading respiration rhythm and the concentration signal of emitted CO2 is inserted into a pocket 55. In particular, at the mouth and the nose, the facial support 40 has a concave and protruding central part 53, in which the pocket 55 which houses the sensor 23 is formed. The gas exhaled by the patient can accumulate inside the central part 53 and the respiration rhythm and concentration of the expired carbon dioxide can be measured by the sensor 23, as described below. The facial support 40, supported by straps 57 which are adjustable on the head of the patient 1, is provided with ocular openings 56 to allow a correct vision by the patient and the walls of the central part 53 are provided with suitable ventilation inlets 54 to allow the patient to breathe correctly.

As illustrated in FIGS. 6,7, the electrodes 42-47 are connected to the sensor 20 by means of conductors 60 incorporated in the thickness of the facial support 40 and by means of electrodes 71 received in the pocket 41. Similarly, the electrodes 49 and 50 are connected to the sensor 21 by conductors 61 also incorporated in the thickness of the facial support 40 and by electrodes 76 received in the pocket 48.

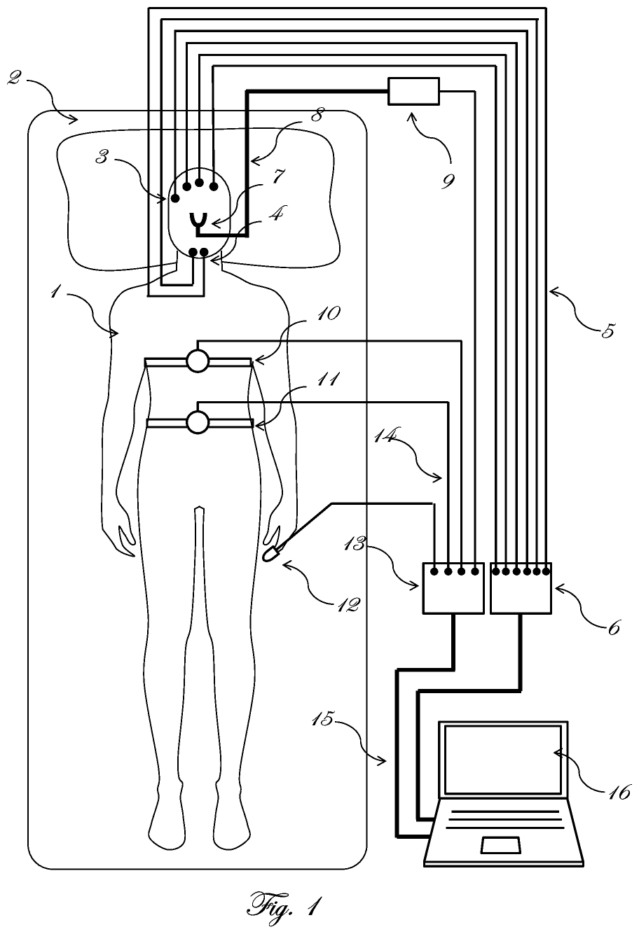

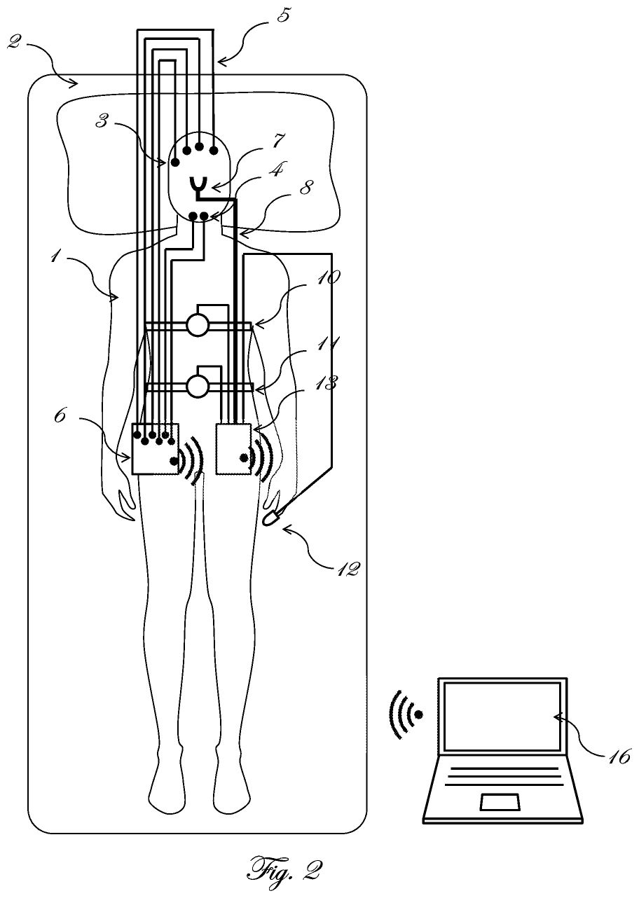

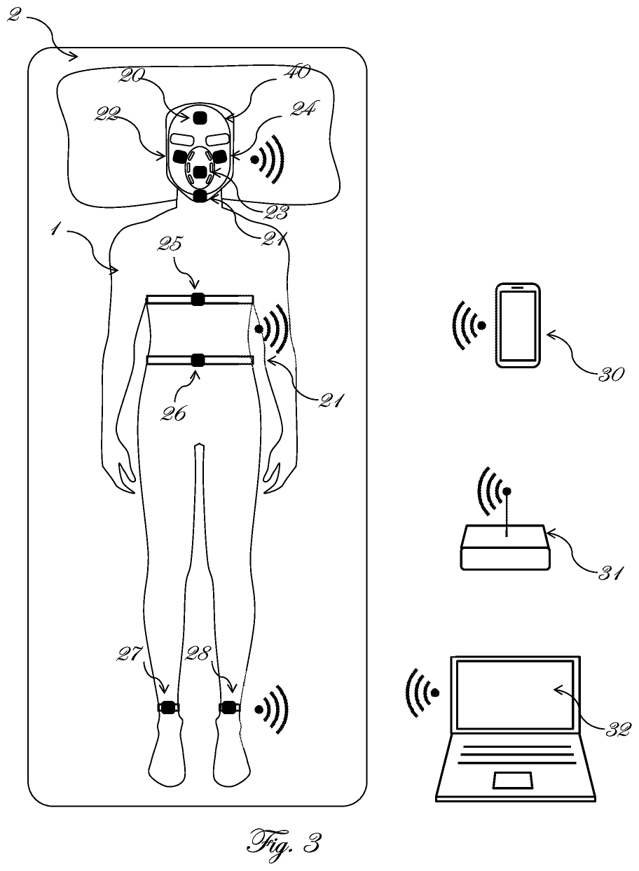

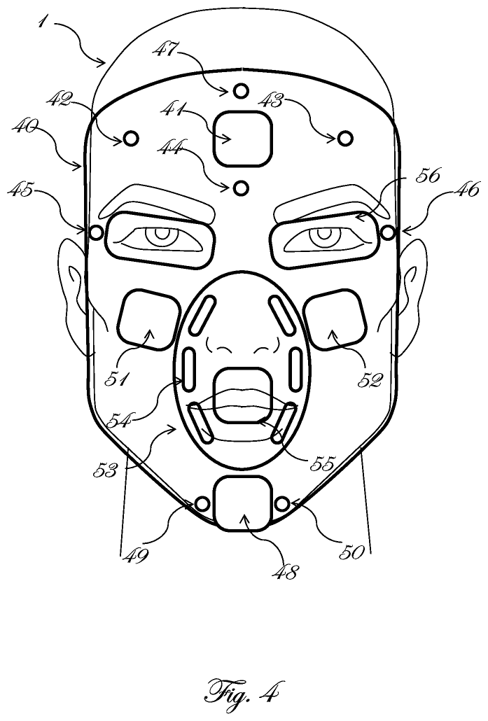

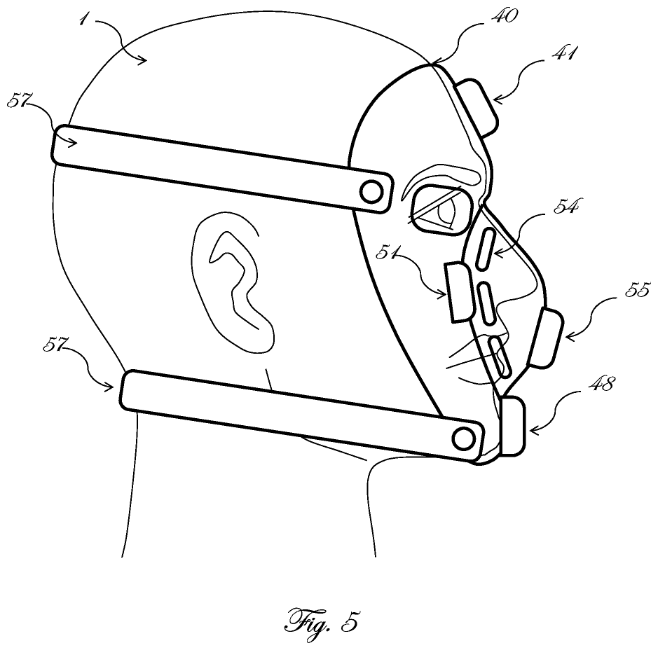

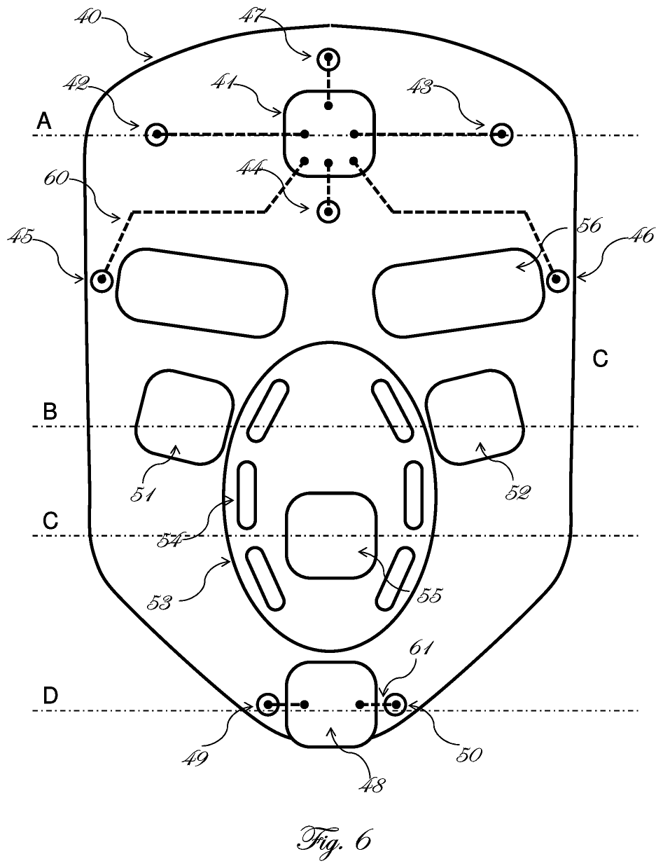

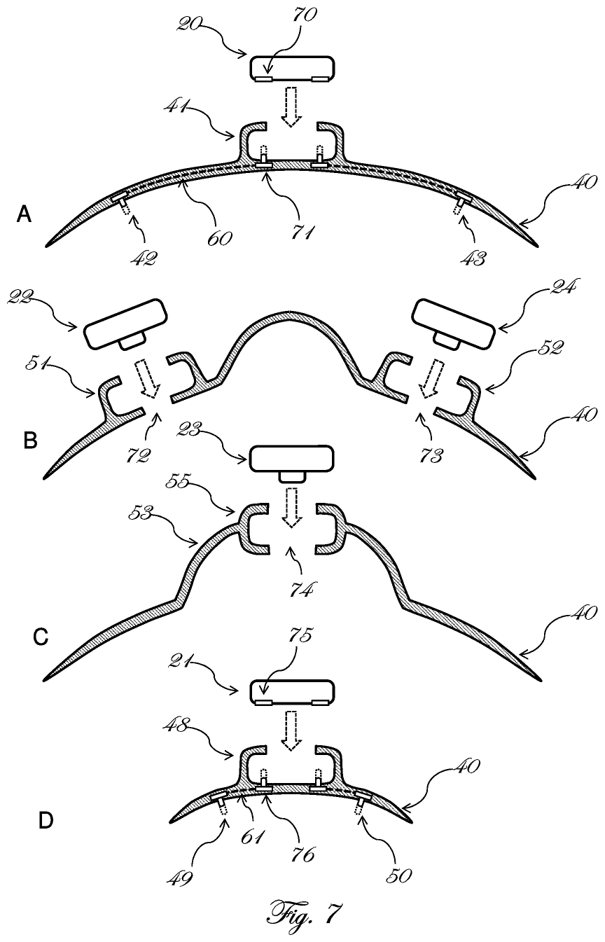

FIG. 7 represents four section views, indicated by A,B,C,D, of the facial support 40 in accordance with the corresponding section lines A,B,C,D of FIG. 6.

In particular, view A shows in section the pocket 41 of the sensor 20 for measuring the EEG/EOG signals. This sensor 20 is provided with electrodes 70 and once inserted into the pocket 41 comes into electric contact with the electrodes 71, which are provided with an elastically retractable contact tip to ensure a secure contact. The electrodes 42-47 are also provided with an elastically retractable tip. While the conductors 60 are completely embedded in the thickness of the facial support 40, the electrodes 42-47 and 71 are only partially embedded so as to be mechanically supported, but at the same time the electrodes 42-47 are electrically exposed to the patient's skin and the electrodes 71 to the electrodes 70 of the sensor 20.

View B shows in section the pockets 51 and 52, the oximeter sensor 22 and the sensor 24 for reading respiratory sound and for reproducing sound or voice messages. The aforesaid pockets have openings 72 and 73, so as to allow the sensor 22 to face its optical reading windows towards the patient's face, and the sensor 24 to be in mechanical contact with patient's face, respectively.

View C shows in section the central part 53 and the pocket 55 of the sensor 23 for measuring respiratory flow and concentration of CO2 emitted. The pocket 55 is provided with a suitable opening 74 so as to expose the CO2 detecting part of the sensor 23 to the aforementioned oro-nasal cavity.

View D shows in section the pocket 48 of the sensor 21 for measuring the EMG signal. This sensor 20 is provided with electrodes 75 and once inserted into the pocket 48 it comes into electric contact with the electrodes 76, which are provided with an elastically retractable contact tip to ensure a secure contact. While the conductors 61 are completely embedded in the thickness of the facial support 40, the electrodes 49 and 76 are only partially embedded so as to be mechanically supported, but at the same time electrically exposed, the electrodes 49 to the patient's skin and the electrodes 76 to the electrodes 75 of the sensor 21.

The described electrodes are made of a conductive material compatible with human skin and in the operative phase a conductive gel can be applied to them.

FIG. 8 shows the sensor 20 for measuring the EEG/EOG signals, together with its block circuit diagram thereof. An energy accumulator BA and a module BM for managing the charge/discharge of the energy accumulator BA, which supplies energy to the other blocks of the same circuit, are highlighted. The contacts for recharging the accumulator BA are indicated with BA− and BA+. The EEG signal, collected by the electrodes 42,43,44, reaches the inputs F7,F8,FpZ respectively, and the EOG signal, collected by the electrodes 45,46,47, reaches the input AT1,AT2,FZ respectively. The EEG and EOG signals are amplified and then converted by respective analogue-digital converters ADC to be numerically processed by a microcontroller MCU. In turn, the microcontroller MCU is connected to a radio transceiver RF which communicates the EEG and EOG data sampled over time to the outside.

FIG. 9 shows the sensor 21 for measuring the EMG signal, together with its block circuit diagram. The energy accumulator BA and the charge/discharge management module BM are highlighted. The EMG signal, collected by the electrodes 49,50, reaches the inlets L and R respectively, is amplified and then converted by the respective analogue-digital converters ADC to be numerically processed by the microcontroller MCU which transmits via the radio transceiver RF the EMG data sampled over time to the outside.

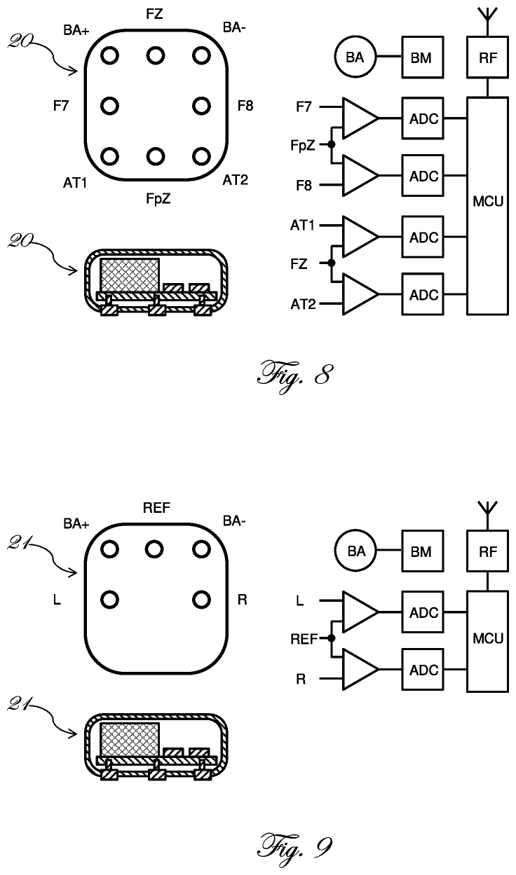

FIG. 10 shows the sensor 23 for measuring respiratory rate and concentration of CO2 emitted, together with its block circuit diagram. In addition to the energy accumulator BA and to the charge/discharge management module BM, the blocks relating to the respiratory rate detector T are also represented, made for example by means of a thermocouple or a pressure transducer, and the CO2 detector, made for example by means of NDIR (non-dispersive infrared) or EC (electrochemical) or MOS (metal oxide semiconductor) technology. The signals produced by the aforesaid detectors, suitably processed and numerically converted by circuits not shown in the figure, are passed to the microcontroller MCU which transmits via the radio transceiver RF the data sampled over time to the outside. The sensor 23 is provided with an opening 100 to allow the detector T and the detector of CO2 to be exposed to the air exhaled by the patient.

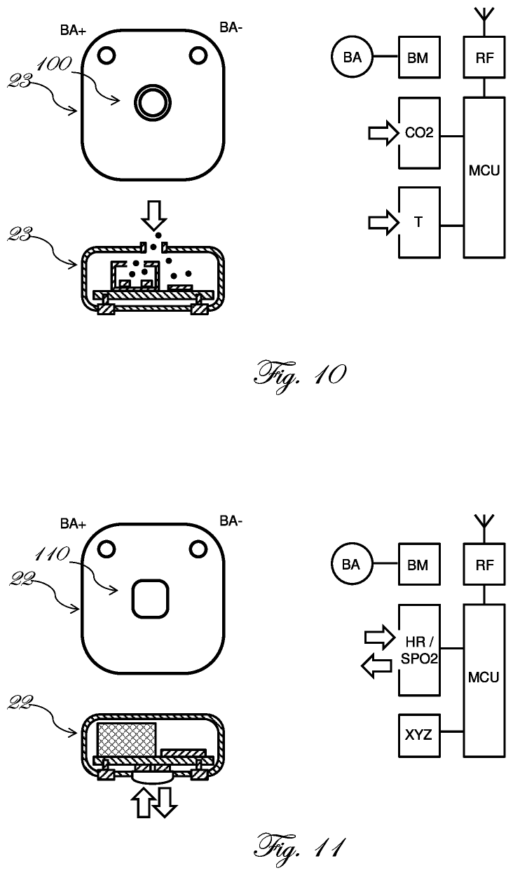

FIG. 11 shows the sensor 22 for measuring heart rate (HR), blood oxygen saturation (SPO2) and movement (XYZ), together with its block circuit diagram. In addition to the energy accumulator BA and the charge/discharge management module BM, there are also represented the blocks relating to the detector of heart rate and oxygen saturation in the blood (HR/SPO2), realized for example by means of an optical pulse oximetry detector, and the movement detector (XYZ), realized for example by means of an MEMS (micro-electro-mechanical system) accelerometer. The signals produced by the aforesaid detectors, suitably processed and numerically converted by circuits not shown in the figure, are transferred to the microcontroller MCU, which transmits via the radio transceiver RF the data sampled over time to the outside. The sensor 22 is provided with a window 110 to allow the detector of HR and of SPO2 to be exposed to the patient's subcutaneous blood vessels.

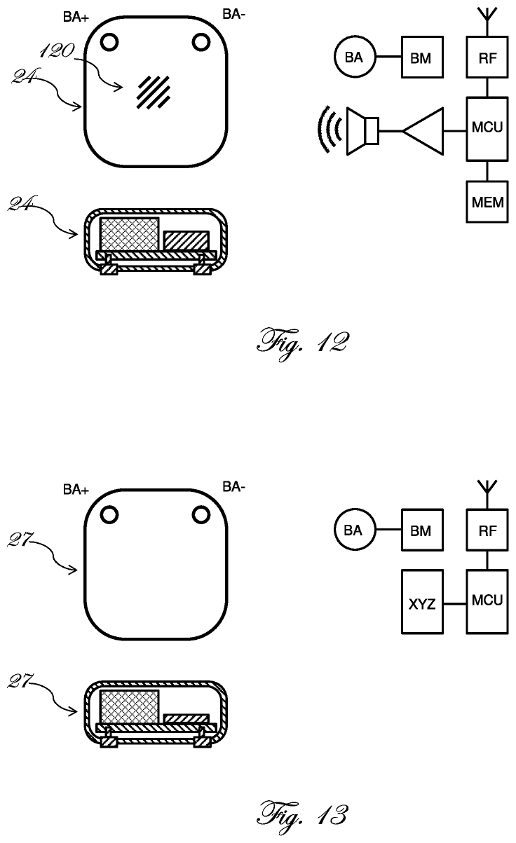

FIG. 12 shows the sensor 24 for reading respiratory sound and for reproducing sound or voice messages, together with its block circuit diagram. In addition to the energy accumulator BA and to the charge/discharge management module BM, the blocks are also represented relating to the microcontroller MCU which generates audio signals, obtained by suitable waveform algorithms or read by a solid-state memory MEM, which are then suitably amplified and reproduced by a loudspeaker or by a bone transducer. The mode and type of messages are managed by an external device, with which the sensor 24 communicates via the radio transceiver RF connected to the microcontroller MCU. The sensor 24 is provided with a grid 120 by means of which the sound is transmitted to the patient.

FIG. 13 shows the sensor 27 for measuring movement (XYZ), together with its block circuit diagram. In addition to the energy accumulator BA and to the charge/discharge management module BM, the blocks relating to the movement detector (XYZ) are also represented, made for example by means of an accelerometer MEMS (micro-electro-mechanical system). The signal produced by the movement detector, suitably processed and numerically converted by circuits not shown in the figure, is passed to the microcontroller MCU which transmits via the radio transceiver RF the data sampled over time to the outside.

The biomedical parameters monitoring system illustrated in FIG. 3 has the advantage of not requiring any wiring and therefore avoids all the drawbacks seen in the introduction, which lead to provide incorrect data and therefore to distort the diagnosis.

Reading the concentration data of CO2 emitted makes the biomedical parameters monitoring system of FIG. 3 more efficient from a diagnostic point of view compared to the known systems.

The use of a facial support on which the most important sensors are concentrated makes the system less invasive and annoying than the known systems. Variations are possible in the configuration of the facial device and in the number and arrangement of the sensors.

1. Biomedical parameters monitoring system for the diagnosis of sleep disorders, including a plurality of sensors reading a series of biometric parameters and one or more external devices collecting the data read by the sensors to make the diagnosis, wherein it includes a facial support on which at least one sensor is mounted for reading the electroencephalographic signal and the electrooculography signal and a sensor for reading the electromyography signal, in which the facial support has a central part, suitable for being located at the mouth and the nose, on which a sensor is mounted to read the respiration rhythm and the signal of the carbon dioxide emitted, wherein each of the sensors is provided with a radio transceiver to transmit the reading data to said one or more external devices.

2. Monitoring system according to claim 1, wherein the facial support has a mask configuration.

3. Monitoring system according to claim 1, wherein the central part of the facial support is concave and protruding.

4. Monitoring system according to claim 1, wherein the walls of the central part of the facial support are provided with ventilation inlets.

5. Monitoring system according to claim 1, wherein a sensor for measuring heart rate, blood oxygen saturation and movement is mounted on the facial support.

6. Monitoring system according to claim 1, wherein a sensor for reading respiratory sound and for reproducing sound or voice messages is also mounted on the facial support.

7. Monitoring system according to claim 1, including a sensor for measuring thoracic expansion and a sensor for measuring abdominal expansion.

8. Monitoring system according to claim 1, including sensors for measuring movement of the limbs.

9. Monitoring system according to claim 1, wherein pockets are formed in the facial support, inside which the sensors mounted on the facial support are received.

10. Monitoring system according to claim 1, wherein each of the sensors includes an energy accumulator and a management module for charging/discharging the energy accumulator to supply energy to the sensor components, wherein inside the sensor the detected signal is amplified, converted by an analog-digital converter, and sent to a microcontroller connected to the radio transceiver which communicates the data sampled over time to the external device.

11. Monitoring system according to claim 1, wherein the sensor for reading the electroencephalographic signal and the electrooculography signal and the sensor for reading the electromyography signal are connected to respective electrodes mounted on the facial support, the connection between sensors and electrodes being achieved by conductors incorporated into the facial support.

12. Monitoring system according to claim 11, wherein the electrodes are each provided with an elastically retractable contact tip.

13. Monitoring system according to claim 1, wherein the facial support is made of soft elastic material.

14. Monitoring system according to claim 1, wherein the facial support is provided with adjustable straps.
